(19) United States
(12) Patent Application Publication
(10) Pub. No.: US 2020/0222451 A1
(43) Pub. Date: Jul. 16, 2020

(54) Composition

(71) Applicant: Optibiotix Limited, York Yorkshire (GB)

(72) Inventors: Stephen Patrick O'Hara, Yorkshire (GB); Sofia Kolida, Yorkshire (GB)

(21) Appl. No.: 16/630,941

(22) PCT Filed: Jul. 27, 2018
(86) PCT No.: PCT/GB2018/052147

§ 371 Date: Jan. 14, 2020

(30) Foreign Application Priority Data
Jul. 28, 2017 (GB) 1712151.8

(51) Int. Cl. A61K 33/24; A61K 31/736; A61K 31/702; A61K 9/00; A61K 45/06

(52) U.S. Cl. CPC A61K 33/24; A61K 31/736; A61K 45/06; A61K 9/0056; A61K 31/702

(57) ABSTRACT

The present invention relates to compositions and foodstuffs for use in increasing gastrointestinal microbiota diversity comprising: a) glucomannan; b) chromium; and c) a prebiotic (FOS). The compositions and foodstuffs are particularly suited for that increasing gastrointestinal microbiota diversity over a prolonged period of time which can assist individuals in reducing their weight and, more importantly, maintaining their lower weight over the longer term.

### TECHNICAL FIELD OF THE INVENTION

The invention relates to compositions and formulations for use in increasing gastrointestinal microbiota diversity. Such compositions are particularly useful in sustaining and/or managing weight loss over a prolonged period of time.

### BACKGROUND TO THE INVENTION

Overweight and obese are conditions defined as abnormal or excessive fat accumulation that may impair health. It results from imbalances in the body's regulation of energy intake, expenditure and storage.

Obesity is one of the greatest public health challenges of the 21st century. It is a complex condition, one with serious social and psychological dimensions, that affects virtually all age and socioeconomic groups in both developed and developing countries. The health consequences of obesity range from increased risk of premature death to serious chronic conditions that reduce the overall quality of life. Excess weight is the fifth leading risk for global deaths. At least 2.8 million adults die each year as a result of being overweight or obese. In addition, 44% of the diabetes burden, 23% of the ischaemic heart disease burden and between 7% and 41% of certain cancer burdens are attributable to being overweight or obese.

Among the reasons that have led to the dramatic increase in obesity are an increase in the intake of high fat, salt and refined sugar foods that are energy rich but otherwise low in nutritional value (vitamins, minerals, micronutrients) in combination with an increasingly sedentary nature to everyday life with a dramatic decrease in physical activity.

Prebiotics have attracted interests as candidate compounds for the control of obesity and associated metabolic disorders. In animal studies, prebiotics have been shown to regulate the intake of food, prevent weight gain, beneficially alter lipid metabolism and reduce obesity-related inflammation. So far, most studies have focused on simply supplementing the diet with inulin and fructooligosaccharides (FOS).

WO2015/067936 discloses compositions for weight management comprising: a) a microbiome modifying component; b) a satiety modifying component; and c) a metabolic modifying component.

Griffin et. al., (2017) Cell Host & Microbe, 21, 1-13, January 11, discloses how dietary practices can alter bacterial diversity in the human gut microbiota and that dispersal of diet-responsive bacterial taxa can enhance subsequent responses to diet interventions.

It is an object of the present invention to provide a formulation which can be used for increasing gastrointestinal microbiota diversity. It would be beneficial if the formulation provided could be used for weight management—whether as a food supplement, additive or potential treatment. It is a further object of the present invention to provide a formulation which can reduce or modulate weight gain—especially for modulating and maintaining weight loss over a prolonged period of time. It would also be preferable that the formulation could be easily administered as either a medicament or a food supplement or a calorie restricted meal replacement product.

### SUMMARY OF THE INVENTION

In accordance with a first aspect of the present invention, there is provided a composition for use in increasing gastrointestinal microbiota diversity comprising:

a) glucomannan;

b) chromium; and

c) a prebiotic.

The term “chromium” covers all dietary chromium salts, including trivalent chromium (Cr(III) or Cr³⁺) which naturally occurs in trace amounts in foods and waters. Preferably, the chromium salt comprises chromium picolinate.

The term “prebiotic” is intended to mean a selectively fermented ingredient that allows specific changes, both in the composition and/or activity in the gastrointestinal microflora flora that confers benefits upon host wellbeing and health.

Preferably the prebiotic comprises one or more selected from: inulin, fructooligosaccharides (FOS), galactooligosaccharides (GOS), α-gluco-oligosaccharides and β-glucans and combinations thereof. More preferably, the prebiotic comprises fructans including fructo-oligosaccharides (FOS) and inulin and combinations of fructo-oligosaccharides and inulin. Most preferably, the prebiotic comprises fructans having a high oligofructose content of up to 50%, 55%, 60%, 65%, 70%, 75%, 80%, 85%, 90% or 95%. The composition may comprise glucomannan in the range of about 0.6 g to about 1.4 g; chromium in the range of about 60 μg to about 100 μg; and/or prebiotic in the range of about 1.5 g to about 4.0 g. More preferably, the composition may comprise glucomannan in the range of about 0.8 g to about 1.2 g; chromium in the range of about 70 μg to about 90 μg; and/or prebiotic in the range of about 0.5 g to about 2.5 g. Even more preferably, the composition comprises glucomannan present at about 1 g; chromium present at about 79 μg; and/or prebiotic present in the range of about 1 g to about 2 g.

The glucomannan may be agglomerated with other components such as with a carrier such as maltodextrin or dextran to improve dispersion, increase solubility and enhance the satiety effect.

The composition may be formulated in a number of ways, but it is preferred that it is in the form of a food stuff or food additive, a dietary supplement (i.e. the composition could be blended with foods/drinks or consumed alongside foods/drinks), or a meal replacement drink or snack bar.

The composition may be for use in increasing gastrointestinal microbiota diversity over a prolonged period of time. That is to say that it increases gastrointestinal microbiota diversity by up to 1 week, 1 month, 3 months or more. It has been surprisingly identified by the present inventors that increasing gastrointestinal microbiota diversity over a prolonged period of time can assist with individuals reducing their weight and, more importantly, maintaining their lower weight over the longer term.

It will be apparent to the skilled addressee that the composition may also be in any easily administered form, for example in the form of a powder, tablet, or capsule. The composition may further comprise an excipient or carrier compound to modify the release profile of one or more of the components through the intestinal environment.

The composition may further comprise one or more active ingredients selected from: vitamins, minerals, phytochemicals, antioxidants, and combinations thereof.

Vitamins may include fat soluble vitamins such as vitamin A, vitamin D, vitamin E, and vitamin and combinations thereof. In some embodiments, vitamins can include water soluble vitamins such as vitamin C (ascorbic acid), the B vitamins (thiamine or B1, riboflavoin or B25 niacin or B3, pyridoxine or B6, folic acid or B9, cyanocobalimin or B12, pantothenic acid, biotin), and combinations thereof.

Minerals may include, but are not limited to, sodium, magnesium, chromium, iodine, iron, manganese, calcium, copper, fluoride, potassium, phosphorous, molybdenum, selenium, zinc, and combinations thereof.

Antioxidants may include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, fiavonoids, and combinations thereof.

Phytochemicals may include but are not limited to cartotenoids, chlorophyll, chlorophyllin, fiber, flavanoids, anthocyamns, cyaniding, delphinidin, malvidin, pelargonidin, peonidin, petunidin, flavanols, catechin, epicatechin, epigallocatechin, epigailocatechingallate, theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof.

In accordance with another (but related) aspect of the present invention, there is provided a functional foodstuff for use in increasing gastrointestinal microbiota diversity comprising:

a) glucomannan;

b) chromium; and

c) a prebiotic.

The prebiotic preferably comprises fructooligosaccharides (FOS) and inulin and combinations of fructooligosaccharides (FOS) and inulin.

Preferably, the foodstuff comprises glucomannan in the range of about 0.6 g to about 1.4 g; chromium in the range of about 60 μg to about 100 μg; and/or prebiotic in the range of about 1.0 g to about 4.0 g. More preferably, the foodstuff comprises glucomannan in the range of about 0.8 g to about 1.2 g; chromium in the range of about 70 μg to about 90 μg; and/or prebiotic in the range of about 1.0 g to about 2.5 g. Most preferably, the foodstuff comprises glucomannan present at about 1 g; chromium present at about 79 μg; and/or prebiotic present in the range of about 1 to about 2 g.

The foodstuff may be in the form of as a dietary supplement, a meal replacement drink or snack bar. If the foodstuff is in the form or a meal replacement drink or snack bar, it may be formulated with one or more of the following ingredients: whey, skimmed milk powder, oat flour, MCT powder, soya or sunflower lecithin, flaxseed, chia seed, xanthan gum, flavourings, natural and/or artificial sweeteners (for example Stevia), vitamins and minerals, superfoods and extracts thereof, natural and/or artificial flavourings and/or colorants and probiotics

The foodstuff may be for use in increasing gastrointestinal microbiota diversity over a prolonged period of time.

The foodstuff may further comprise active ingredients selected from: vitamins, minerals, phytochemicals, antioxidants, and combinations thereof.

The composition as herein above described and/or foodstuff as hereinabove described, may be taken up to three or four times a day as part of a calorie controlled diet regime. The composition or foodstuff may be taken during and/or after a reduced calorie intake diet regime. Such a calorie controlled diet regime may comprise consuming up to four products over the course of the day in order to replace two out of three daily meals. The four products may be in the form of two meal replacement drinks and two mid-meal snack bars. The two meal replacement drinks may include two thirds of the daily amount, whereas the two mid-meal snack bars would collectively include one third of the daily amount.

The composition or foodstuff may comprise glucomannan in a daily dose in the range of about 1.8 g to about 4.2 g; chromium in a daily dose in the range of about 180 μg to about 300 μg; and/or prebiotic in a daily dose in the range of about 1.0 g to about 12 g.

The composition or foodstuff may comprise glucomannan in a daily dose in the range of about 2.4 g to about 3.6 g; chromium in a daily dose in the range of about 210 μg to about 270 μg; and/or prebiotic in a daily dose the range of about 1.5 g to about 12 g.

The composition or foodstuff comprises glucomannan in a daily dose of about 3 g; chromium in a daily dose of about 79 μg; and/or prebiotic in a daily dose in the range of about 2.0 g to 12 g.

In accordance with another aspect of the present invention, there is provided a method of promoting or maintaining weight loss in an individual, the method comprising administering the composition of foodstuff as herein above described. The composition or foodstuff may be administered up to 3 times a day. Preferably, the composition or foodstuff is administered before, during (or as part of) or after a reduced calorie intake diet regime.

In accordance with another aspect of the present invention, there is provided a use of a composition as herein above, in the manufacture of a medicament for the treatment of obesity.

### DETAILED DESCRIPTION OF THE INVENTION

Embodiments of the present invention will now be described, by way of example only with reference to the following figures and examples detailed below.

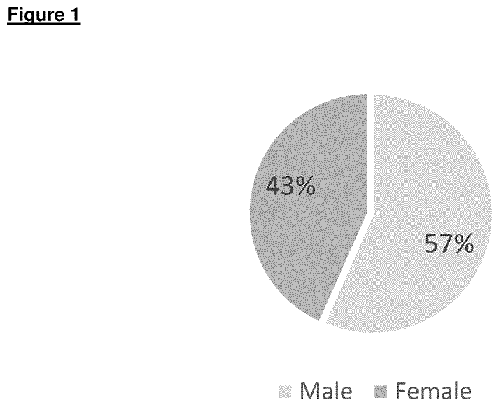

FIG. 1 is a graph illustrating the results of survey data in Example 2, showing the gender percentage of purchasers of the calorie restricted meal replacement range;

FIG. 2 is a graph illustrating the results of survey data in Example 2, showing the broad age range of respondents;

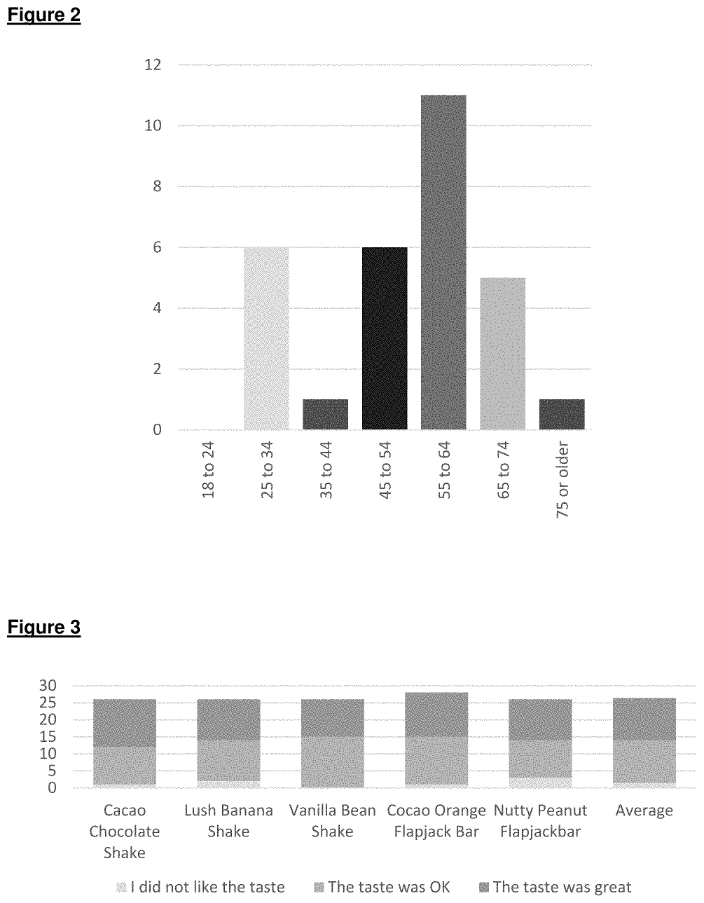

FIG. 3 is a graph illustrating the results of survey data in Example 2, showing consumer responses to the taste of the calorie restricted meal products;

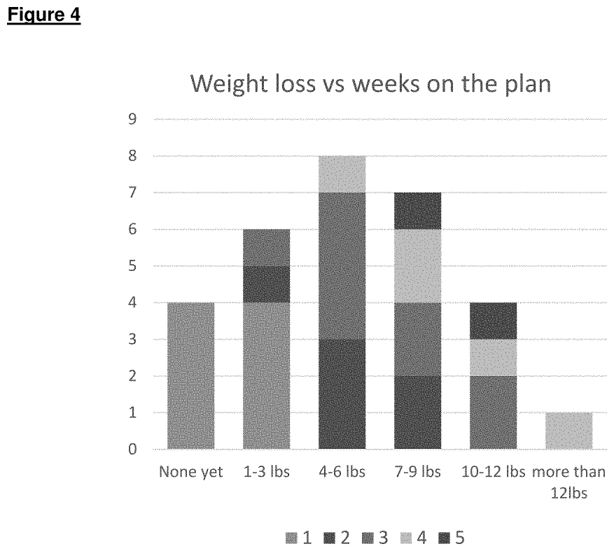

FIG. 4 is a graph illustrating the results of survey data in Example 2, showing consumer responses to the amount of weight they have lost vs weeks on the plan using the calorie restricted meal products;

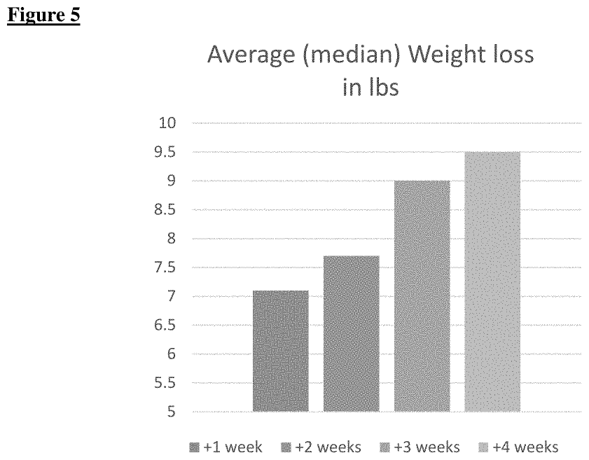

FIG. 5 is a graph illustrating the results of survey data in Example 2, showing consumer responses to the amount of weight they have lost using the calorie restricted meal products;

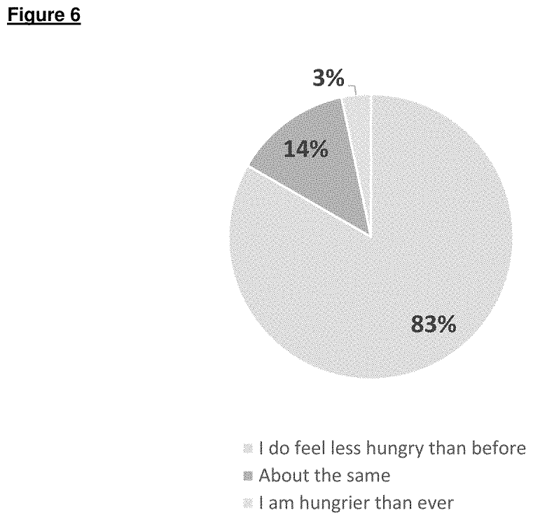

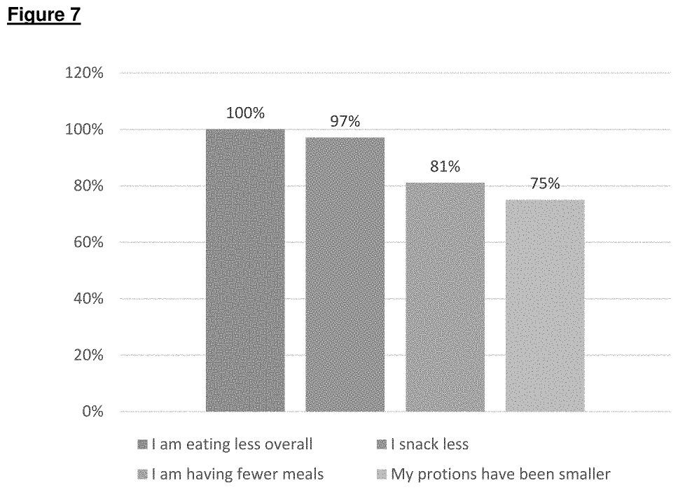

FIG. 6 is a graph illustrating the results of survey data in Example 2, showing consumer responses to how hungry the responders felt whilst using the calorie restricted meal products;

FIG. 7 is a graph illustrating the results of survey data in Example 2, showing consumer responses to how using the calorie restricted meal products had helped responders to consume less food overall and snacking less; and

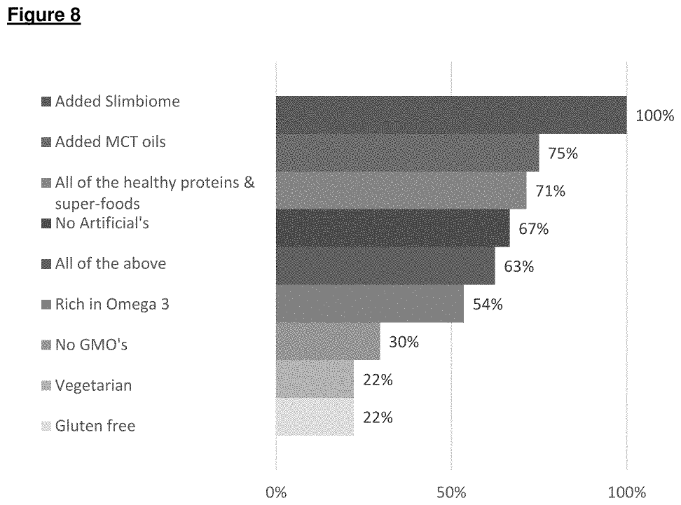

FIG. 8 is a graph illustrating the results of survey data in Example 2, showing consumer responses to how positively responders felt whilst using the calorie restricted meal products in comparison to other weight loss products or health foods.

### EXAMPLES

### Example 1

### SlimBiome® Formulation

A ‘SlimBiome’ formulation including the following components was prepared in accordance with Table 1 and placed in a sachet.

TABLE 1

| Ingredient | mg per sachet | % bst |
|---|---|---|
| GLUCOMANNAN | 1000 | 20.0 |
| CHROMAX ® | 0.633 | 0.01 |
| FOS Beneo P95 | 2000 | 40.0 |
| Citric acid | 125 | 2.5 |
| Maltodextrin | 1486 | 29.7 |
| Ace flavor | 100 | 2.0 |
| Orange flavor | 125 | 2.5 |
| Silicon dioxide | 50 | 1.0 |
| Sucralose | 13.5 | 0.27 |
| betacarotene 1% | 100 | 2.0 |
| TOTAL | 5000 | 100 |

The glucomannan component is marketed under Glucomannan 90% (E-425i) and sourced from Cambridge Commodities Limited, 203 Lancaster Way Business Park, Ely, Cambridgeshire, CB6 3NX, UK.

CHROMAX® is the chromium component and sourced from Ingredia Nutritional S. A., 51, Avenue Fernand Lobeddez, CS, 60946, 62033 Arras Cedex, France and has a specification of 12 - 12.5% of chromium. CHROMAX® is salt of chromium and picolinic acid in powder, with a standardized content of 12-12.5% pure chromium. Picolinic acid is a natural tryptophan metabolite making it highly bioavailable.

The FOS Beneo P95 component was sourced from BENEO GmbH Maximilianstrasse 10, 68165 Mannheim, Germany. The oligofructose (fructo-oligosaccharide, FOS) are composed of fructose units linked by a beta-1,2-bond, partly with an end-standing glucose unit. The degree of polymerisation (=number of monosaccharide units) ranges between 2 and 8 and is formed by partial enzymatic hydrolysis of chicory inulin. It has an oligofructose content of 95% and a sweetness level of 30% and is a powder form.

A placebo formulation was also prepared without the active ingredients of glucomannan, chromium and FOS, and the components shown in Table 2 below, and this formulation inserted into a sachet.

TABLE 2

| Ingredient | mg per sachet | % bst |
|---|---|---|
| Citric acid | 125 | 6.3 |
| Maltodextrin | 1486 | 74.3 |
| Ace flavor | 100 | 5.0 |
| Orange flavor | 125 | 6.3 |
| Silicon dioxide | 50 | 2.5 |
| Sucralose | 13.5 | 0.68 |
| betacarotene 1% | 100 | 5.0 |
| TOTAL | 2000 | 100 |

The contents of the sachets were then blended with other ingredients to produce calorie restricted powdered meal replacement shake or a meal replacement snack bar.

For the powdered meal replacement shake or snack bar, the sachet was blended with the following ingredients: whey, skimmed milk powder, oat flour, MCT powder, soya or sunflower lecithin, flaxseed, chia seed, xanthan gum, flavourings, natural and/or artificial sweeteners (for example Stevia), vitamins and minerals, superfoods and extracts thereof, natural and/or artificial flavourings and/or colorants and probiotics. Other ingredients which are specific to the meal replacement may be included, such as vanilla pods or raw or processed cocoa.

### Example 2

### Customer Survey

A customer survey was commissioned in order to gain feedback from customers who had bought calorie restricted meal replacement products including the SlimBiome® formulation. The specific objectives of the survey were to understand the effectiveness of the meal replacements, the likability of the products & flavours and the appeal of specific ingredients.

The customer survey was conducted between July and August 2016. 127 customers were contacted who had bought the product directly. The only recruitment criteria set was the customers had purchase of the product directly via the official sales website and as such the respondents were free living adults, with no specified weight range, health status or dietary restriction, so as to replicate a human intervention study.

The survey was conducted in two phases: phase 1-27^{th} July 2016—87 contacted; and phase 2—18^{th} August 2016—40 contacted. Personal information collected was limited to age and gender. No details were obtained on the respondents as to existing physical health condition, eating or exercise habits.

### Summary of Findings

Overall, response to the calorie restricted meal replacement range was positive with the majority of respondents losing weight during the period they had been using the products and liking the taste of the products and finding the active ingredients as a motivating benefit. Verbatim comments suggest that respondents were open to more flavours and formats in the range, but many had managed to stick with the calorie restricted meal replacement range plan unlike other plans. Some respondents stated that they enjoyed the diet which wasn't normal for them as they had previously not stuck to diets, whilst others mentioned that they were never hungry.

Effectiveness of the calorie restricted meal replacement range

100% of those on the plan for more than 1 week stated that they had lost weight. For the 53% of respondents who had been on the plan for 3 weeks or more, the median weight loss was 7-9 lbs (3.175-4.082 kgs) in total. On average, the longer the respondents were on the plan the greater weight loss recorded (i.e. those on the plan for 3 weeks lost more on average than those on the plan for 2 weeks). Respondents were only asked to record their total weight loss at the time the survey was conducted).

83% of respondents agreed with the statement that “I do feel less hungry than before”, 100% of respondents claim that “I am eating less overall”, 97% of respondents claim that “I snack less”.

The addition of Slimbiome® formulation “really appealed” to respondents.

With reference to FIG. 1, it can be seen that the restricted meal replacement range was bought by both women (57%) and men (43%).

With reference to FIG. 2, the survey attracted a broad age range of respondents of people aged from 25 to over 75. The largest single group of respondents 37% were aged between 55 and 64, with 73% of respondents aged between 45 to74.

In respect of FIG. 3, consumer responses to the taste of the products was positive. When asked what products from the calorie restricted meal replacement range had they tried and of those they had tried and how did they find the taste, consumer responses to the taste of the products was positive. 95% of respondents agreed with the comments that either “the taste was great” or “the taste was OK”. 47% of respondents agreed with the comment “the taste was great”. Response across the product formats and flavours was broadly similar.

With reference to FIG. 4, the calorie restricted meal replacement products were working with respondents claiming they have lost weight. When questioned how long have they been on the calorie restricted meal replacement plan and how much weight have they lost, 100% of those on the plan for more than 1 week have lost weight. For those who had been on the plan for 3 weeks or more (53% of respondents) the median weight loss over that period was 7-9 lbs (3.175-4.082 kgs) in total.

With reference to FIG. 5, using mid-point analysis, approximate averages can be calculated (i.e. 5 lbs (2.267 kg) for range 4-6 lbs (1.814-2.721 kgs)). When questioned how long had they been on the calorie restricted meal replacement plan and how much weight have you lost, the average weight loss for all 30 respondents was 5.5 lbs (2.494 kgs). For the 73% of respondents that had been on the plan for more than 1 week the average weight loss was 7.1 lbs (3.220 kgs). On average, the longer the respondents were on the plan the greater weight loss recorded (i.e. those on the plan for 3 weeks lost more on average than those on the plan for 2 weeks).

With reference to FIG. 6, the calorie restricted meal replacement plan seemed to be positively helping people feel less hungry. When asked whether eating the calorie restricted meal replacement products have helped reduce how hungry you feel, 83% of respondents agreed with the statement that “I do feel less hungry than before”, with only respondent (3%) claimed that “I am hungrier than ever” (this respondent had been on the plan for 2 weeks and had lost 7-9 lbs (3.175-4.082 kgs)).

With reference to FIG. 7, the calorie restricted meal replacement product plan was also helping people consume less food overall and snacking less. When asked whether eating the shakes and bars had helped responders reduce their overall food intake, 100% of respondents claimed that they were “eating less overall”. 97% of respondents claimed that they “snacked less”, 81% of respondents stated they are having fewer meals and 75% of respondents stated their food portions were smaller.

With reference to FIG. 8, the Slimbiome® formulation was one of the most important features of the calorie restricted meal replacement products that appealed to respondents. When asked what benefits from the calorie restricted meal replacement product range really appealed, 100% of respondents claimed that “added Slimbiome” really appealed.

### Example 3

### Investigation into the effects of SlimBiome® on appetite, cravings, and food intake in healthy overweight adults (BMI 27.5-32.5 kg/m²) following standard dietetic advice.

The study was carried out to determine the effect of SlimBiome® formulation on satiation (fullness), satiety (hunger/cravings between meals) and food intake/choice. Product tolerance (and side-effects), cognitive ability (in the form of mood/cravings) and the ease of following a following a meal plan/compliance with the meal replacement products incorporating the Slim Biome® formulation was also assessed.

### Study Design

The study was conducted on 40 female subjects aged 21 to 65 years, with a BMI >27.5-32.5 kg/m². The study was in the form of a prospective, randomized, parallel-group trial with two groups: Dietetic advice plus placebo (control) or Dietetic advice plus SlimBiome three times a day (test).

### Inclusion Criteria

To be included in the study, the subjects had to also fulfil the criteria that they: had not followed a calorie restricted diet within the last month and had not lost a significant amount of weight in the previous year; had not increased physical activity levels in the past 2-4 weeks or intending to modify them during the study; were willing, able and likely to comply with all study procedures and restriction including being willing to follow the calorie restricted diet; and were able to eat most everyday foods. Subjects were excluded on the following grounds that they: had significant health problems; were taking medication or supplements known to affect appetite or weight within the past month and/or during the study; were pregnant, planning to become pregnant or breastfeeding; had a history of anaphylaxis to food; had known allergies or intolerance to foods and/or to the study materials (or closely related compounds) or any of their stated ingredients; had a BMI <27.5 kg/m² or >32.5 kg/m²; had reported that they were currently undergoing a calorie restricted diet or having lost significant amount of weight in the previous year; had abnormal eating behaviour; were smokers and those who have recently ceased smoking; had participation in another experimental study or receipt of an investigational drug/product within 30 days of the screening visit; had significantly changed their physical activity in the past 2-4 weeks or who intend to change them during the study; were receiving systemic or local treatment likely to interfere with the evaluation of the study parameters; were on specific food avoidance diets; and had worked in appetite or feeding related areas.

### Product Administration

All participants followed standard dietetic advice supplement and also had access to additional on-line material. They were advised on product storage and use conditions prior to use to ensure product consistency throughout the period of the study. The participants were instructed to take their respective products (containing either the placebo or Slimbiome® formulation) daily with water before each of the three main meals.

### Study Visit Protocol

The following parameters were assessed during the study visit: anthropometric measurements; mood (PANAS) and craving (COEQ) measures; attentional bias (visual probe task); food choice (LFCT); food responsiveness (PoF); wellbeing (PSS and BDI); experience of programme; and experience of appetite.

### Measures and Outcomes

Each participant attended a total of 3 study visits (plus screening). Assessments took take place at T=0, 1, and 4 weeks and the following measurements taken:
Anthropometric: body weight, waist circumference, hip circumference Body mass index (BMI), impedance (body fat)
Glucose levels
Visual Analogue Scale (VAS) questionnaire
   Hunger
   Fullness
   Appetite/Desire to eat
   Prospective consumption
   Satisfaction/meal experience
   Thirst
   Nausea/side affects
Cognitive ability—mood/craving/attentional bias to food cues
Eating behaviour (restrained, external, emotional eater; determined at screening)
Food intake (meal intake -g/kcal and meal duration of evening meal; meal choice—consumption/timing of evening snack bar)
Experience of programme (ease/compliance)
Wellbeing Results Human studies using a random double blind placebo controlled trail that when compared to placebo, chromium picolinate reduces food intake (P <0.0001), hunger levels (P<0.05), and fat cravings (P<0.0001) leading to a decrease body weight (P<0.08). Glucomannan is supported by numerous studies demonstrating its efficacy for weight loss when used along with hypocaloric diets. Both glucomannan and soluble fibres like fructans act by increasing satiety after meals by slowing down carbohydrate absorption. These results would suggest a combination of these ingredients would have an additive or synergistic effect.

### Example 4

### Meal Replacement Snack Bar and Shake Formulations

Example meal replacement snack bar and dehydrated shake formulations were formulated as follows:

### Cacao Chocolate Meal Replacement Snack Bar Formulation:

TABLE 3

| Ingredient | (g) |
|---|---|
| WPC Europro Whey | 2.0 |
| Skimmed Milk Powder | 4.0 |
| Soya Protein | 7.0 |
| Oat Flour | 4.0 |
| Maltodextrin | 11.5 |
| Fructooligosaccharides (Beneo) | 0.5 |
| MCT Powder | 1.5 |
| Soya/Sunflower Lecithin | 3.0 |
| Flaxseed | 0.5 |
| Chia Seed | 0.5 |
| Glucomannan | 1.0 |
| Xanthan Gum | 0.5 |
| Flavouring | 1.0 |
| Stevia | 0.1 |
| Cocoa/Raw Cacao (IMCD) | 2.5 |
| Multivits, Superfoods, Probiotics | 0.4 |
| TOTAL | 40.0 |

### Vanilla Bean and Banana Powdered Meal Replacement Shake Formulation:

TABLE 4

| Ingredient | (g) |
|---|---|
| WPC Europro Whey | 2.00 |
| Skimmed Milk Powder | 4.00 |
| Soya Protein | 7.50 |
| Oat Flour | 4.00 |
| Maltodextrin | 13.00 |
| Fructooligosaccharides (Beneo) | 0.50 |
| MCT Powder | 1.50 |
| Soya/Sunflower Lecithin | 3.00 |
| Flaxseed | 0.50 |
| Chia Seed | 0.50 |
| Glucomannan | 1.00 |
| Xanthan Gum | 0.50 |
| Flavouring | 1.50 |
| Stevia | 0.10 |
| Multivits, Superfoods, Probiotics | 0.4 |
| TOTAL | 40.0 |

(NB Superfood component includes 0.633 mg of Chromax ®)

As part of a calorie controlled diet regime, an individual would look to consume up to four products over the course of the day in order to replace two out of three daily meals. The four products may be in the form of two meal replacement drinks and two mid-meal snack bars according to the formulas outlined in Tables 3 and 4 above. It will be obvious to the skilled addressee that the formulas may be altered so as to enable the production of differently tasting products, which still have the beneficial effect of increasing the gastrointestinal microbiota diversity and ultimately facilitating weight loss.

### Example 5

Meal replacement snack bar and shake formulations Meal replacement snack bar and dehydrated shake formulations were produced on the basis of the following composition as defined below in Table 5.

TABLE 5

| | | | Dosage | |
|---|---|---|---|---|
| | | | orignial/ | |
| | | | day (3 | Dosage/ |
| | | | servings) | serving |
| Ingredient | Detail | Supplier | gram | gram |
| Glucomannan | Cambridge - | Elementa | 3.000000 | 1.000000 |
| | Glucommanna | Ingredients | | |
| | 90% (E-425i) | | | |
| | Konjac | | | |
| | Flour | | | |
| | P-M2(2)H | | | |
| Prebiotic/ | FOS: Beneo - | KREGLINGE | 6.000000 | 2.000000 |
| Oligofructose | Orafti P95 | REUROPE NV | | |
| Chomium | Chromax - | Ingredia- | 0.000250 | 0.000083 |
| | Chomium | nutritional | | |
| | Picolinate | | | |
| | TOTAL | | | |

### Shakes

A 201 calorie healthy meal replacement vanilla bean shake was formulated with the following ingredients: MCT (medium-chain triglycerides fats) Powder, Skimmed Milk, Gluten Free Oat Flour, Soya Protein, Whey Protein (Milk), Maltodextrin, Natural Flavouring, Glucomannan Formula [Oligofructose, Glucomannan (Konjac root extract), Chromium Picolinate], Emulsifier (Soya Lecithin), Multivitamin Premix (vitamin A, Thiamin (B1), Riboflavin (B2), Niacin (B3), Pantothenic Acid (B5), vitamin B6, Folic Acid, Vitamin B12, Vitamin C, Vitamin D, Vitamin E, Biotin, Calcium, Phosphorus, Potassium, Iron, Zinc, Copper, Iodine, Selenium, Chromium, Manganese, Magnesium, Flaxseed, Chia (Salvia hispanica) Seeds, Greens Powder Premix (Broccoli, Spinach, Watercress, Green Kale, Barley Grass, Alfalfa, Coriander, Basil, Rosemary, Thyme, Yucca Root, Kelp, Avocado), Premix (Spirulina, Chlorella, Baobab fruit pulp, Maca), Thickener (Xanthan Gum), Live Probiotic Bacterial Cultures Complex (Lactobacillus Acidophilus, Bifidobacterium Bifidum), Sweetener (Steviol Glycosides). The shake was formulated with 1 g of glucomannan, 2 g of FOS and 0.083 g chromium.

The shake is intended promote healthy, sustainable weight loss with a marked increase in vitality which are made from real, wholefood ingredients and have all the healthy nutrients your body craves in one delicious tasting liquid meal.

The shakes are intended to be re-hydrated to the correct viscosity and two shakes consumed per day so as to replace two regular meals. Preferably, they are used to replace breakfast and dinner with a healthy meal shake. 1 sachet (40 g) of powder is blended or mixed with 250 ml to 350 ml of water and an additional glass of water or herbal tea taken.

### Bars

A 115 calorie healthy peanut snack bar was formulated with the following ingredients: Chopped dates, gluten free oats (oats, oat bran 19.8%), air roasted peanuts (17.1%), sultanas, blanched peanuts (7.5%), sunflower seeds, vegetable glycerine, [Oligofructose, Glucomannan (Konjac root extract), Chromium Picolinate]. The bar was formulated with 0.5 g of glucomannan, 1 g of FOS and 0.041 g chromium.

An alternative bar is a 115 calorie healthy orange cacao snack bar was formulated with the following ingredients: Gluten free oats (oats, oat bran 25.1%), cashews, chopped dates, sultanas, vegetable glycerine, cacao nibs (4.3%), cacao powder (3.5%), cacao butter (3.5%), oligofructose (fibre), glucomannan (Konjac root extract), natural orange flavouring, chromium picolinate. The bar was formulated with 0.5 g of glucomannan, 1 g of FOS and 0.041 g chromium.

The bars are intended to be a healthy combination of cold pressed fruits, gluten free oats and seeds, with no added sugar, syrups or fillers. They can therefore be taken as a satisfying snack between meals, after exercising, on the way to work or as a delicious treat. Two bars are to be taken daily.

The forgoing embodiments are not intended to limit the scope of the protection afforded by the claims, but rather to describe examples of how the invention may be put into practice.

1. A composition for use in increasing gastrointestinal microbiota diversity comprising:
a) glucomannan;
b) chromium; and
c) a prebiotic.

2. The composition as claimed in claim 1, wherein the prebiotic comprises one or more soluble fibres.

3. The composition as claimed in either claim 1 or 2, wherein the prebiotic comprises one or more selected from: inulin, fructooligosaccharides (FOS), galactooligosaccharides (GOS), α-gluco-oligosaccharides, β-glucans and combinations thereof.

4. The composition as claimed in any preceding claim, wherein the composition comprises glucomannan in the range of 0.6 g to 1.4 g; chromium in the range of 60 pg to 100 μg; and/or prebiotic in the range of 0.25 g to 4.0 g.

5. The composition as claimed in any one of claims 1 to 3, wherein the composition comprises glucomannan in the range of about 0.8 g to about 1.2 g; chromium in the range of about 70 μg to about 90 μg; and/or prebiotic in the range of about 1.0 g to about 2.5 g.

6. The composition as claimed in any one of claims 1 to 3, wherein the composition comprises glucomannan present at about 1 g; chromium present at about 79 μg;
and/or prebiotic present in the range of about 1 g to about 2 g.

7. The composition as claimed in any one of claims 1 to 6, wherein the composition is in the form of a food stuff or food additive.

8. The composition as claimed in any one of claims 1 to 6, wherein the composition is in the form of as a dietary supplement.

9. The composition as claimed in any one of claims 1 to 6, wherein the composition is in the form of a meal replacement drink or snack bar.

10. The composition as claimed in any one of claims 1 to 9, wherein the composition is for use in increasing gastrointestinal microbiota diversity over a prolonged period of time.

11. The composition as claimed in any preceding claims, wherein the composition further comprises one or more active ingredients selected from: vitamins, minerals, phytochemicals, antioxidants, and combinations thereof.

12. A functional foodstuff for use in increasing gastrointestinal microbiota diversity comprising:
a) glucomannan;
b) chromium; and
c) a prebiotic.

13. The foodstuff as claimed in claim 12, wherein the prebiotic comprises one or more soluble fibres.

14. The foodstuff as claimed in claim 12 or 13, wherein the prebiotic comprises one or more selected from: inulin, fructooligosaccharides (FOS), galactooligosaccharides (GOS), α-gluco-oligosaccharides, β-glucans and combinations thereof.

15. The foodstuff as claimed in any one of claims 12 to 14, wherein the foodstuff comprises glucomannan in the range of 0.6 g to 1.4 g; chromium in the range of 60 pg to 100 μg; and/or prebiotic in the range of about 1.0 g to about 4.0 g.

16. The foodstuff as claimed in any one of claims 12 to 14, wherein the foodstuff comprises glucomannan in the range of 0.8 g to 1.2 g; chromium in the range of 70 pg to 90 μg; and/or prebiotic in the range of about 1 g to about 2.5 g.

17. The foodstuff as claimed in any one of claims 12 to 14, wherein the foodstuff comprises glucomannan present at about 1 g; chromium present at about 79 μg;
and/or prebiotic present in the range of about 1 g to about 2 g.

18. The foodstuff as claimed in any one of claims 12 to 17, wherein the foodstuff is in the form of a food stuff or food additive.

19. The foodstuff as claimed in any one of claims 12 to 17, wherein the foodstuff is in the form of as a dietary supplement.

19. The foodstuff as claimed in any one of claims 12 to 17, wherein the foodtsuff is in the form of a meal replacement drink or snack bar.

20. The foodstuff as claimed in any one of claims 12 to 19, wherein the foodstuff is for use in increasing gastrointestinal microbiota diversity over a prolonged period of time.

21. The foodstuff as claimed in any one of claims 12 to 20, further comprising one or more active ingredients selected from: vitamins, minerals, phytochemicals, antioxidants, and combinations thereof.

22. The composition of any one of claims 1 to 11, of foodstuff of any one of claims 12 to 21, taken up to 4 times a day as part of a calorie controlled diet regime.

23. The composition or foodstuff of claim 22, wherein the composition or foodstuff is taken during and/or after a reduced calorie intake diet regime.

24. The composition or foodstuff of claim 22 or 23, wherein the composition or foodstuff comprises glucomannan in a daily dose in the range of 1.8 g to 4.2 g; chromium in a daily dose in the range of 180 pg to 300 μg; and/or prebiotic in a daily dose in the range of 1.0 g to 12 g.

25. The composition or foodstuff of claim 22 or 23, wherein the composition or foodstuff comprises glucomannan in a daily dose in the range of 2.4 g to 3.6 g; chromium in a daily dose in the range of 210 pg to 270 μg; and/or prebiotic in a daily dose the range of 1.5 g to 12 g.

26. The composition or foodstuff of claim 22 or 23, wherein the composition or foodstuff comprises glucomannan in a daily dose of about 3 g; chromium in a daily dose of about 79 μg; and/or prebiotic in a daily dose in the range of about 2 g to about 12 g.

27. A method of promoting or maintaining weight loss in an individual, the method comprising administering the composition of any one of claims 1 to 6 or the foodstuff of any one of claims 11 to 17.

28. The method as claimed in claim 27, wherein the composition or foodstuff is administered up to 4 times a day.

29. The method as claimed in either claim 27 or 28, wherein the composition or foodstuff is administered before, during (or as part of) or after a reduced calorie intake diet regime.
